(19) United States
(12) Patent Application Publication
(10) Pub. No.: US 2026/0248469 A1
(43) Pub. Date: Aug. 27, 2026

(54) COOLING APPARATUS FOR MEDICAL IMAGING SYSTEM, AND MEDICAL IMAGING SYSTEM

(71) Applicant: GE Precision Healthcare LLC, Waukesha, WI (US)

(72) Inventors: Hailiang Liu, Beijing (CN); Caihong Liang, Beijing (CN); Jian Cao, Beijing (CN); Cunli Zhang, Beijing (CN); Shuli Yu, Beijing (CN)

(21) Appl. No.: 19/541,260

(22) Filed: Feb. 16, 2026

(30) Foreign Application Priority Data
Feb. 25, 2025 (CN) 202510209389.9

(51) Int. Cl. A61B 6/03

(52) U.S. Cl. CPC A61B 6/037

(57) ABSTRACT

A cooling apparatus for a medical imaging system is provided. The cooling apparatus includes: a base; a cooling substrate, configured to conduct heat, the cooling substrate including at least one sub-substrate, and the at least one sub-substrate being disposed on the base; and a pipe, configured to transport a cooling liquid, at least a part of the pipe being disposed on the cooling substrate. Therefore, the size of the cooling substrate can be reduced, thereby reducing the amount of a raw material used for manufacturing the cooling substrate, reducing a fault rate during manufacturing and transportation, and helping reduce the overall costs of the cooling apparatus.

### CROSS REFERENCE TO RELATED APPLICATIONS

The present application claims priority and benefit of Chinese Patent Application No. CN 202510209389.9 filed on Feb. 25, 2025, which is incorporated herein by reference in its entirety.

### TECHNICAL FIELD

Embodiments of the present application relate to the field of electromechanics, and in particular, to a cooling apparatus for a medical imaging system, and a medical imaging system.

### BACKGROUND

A large quantity of electronic modules are disposed in medical imaging systems, and the electronic modules generate heat during operation. Using a positron emission tomography-magnetic resonance (PET-MR) imaging system as an example, the PET-MR imaging system is a medical image diagnosis system that combines positron emission tomography (PET) and magnetic resonance imaging (MRI) technologies. The imaging system can simultaneously detect metabolic and structural information in an organism, and has high resolution and sensitivity to soft tissues.

As one of the core components of the PET-MR imaging system, a detector module generates a large amount of heat during operation, resulting in an increase in operating temperature. If the operating temperature of the detector module is excessively high, normal operation and image quality of the PET-MR imaging system will be affected.

Therefore, to enable the medical imaging system to operate normally and ensure imaging quality, it is necessary to regulate the operating temperature of the electronic module in the medical imaging system.

It should be noted that the above introduction of the background is only for the convenience of clearly and completely describing the technical solutions of the present application, and for the convenience of understanding for those skilled in the art.

### SUMMARY OF THE INVENTION

The inventor has found that cooling apparatuses are currently used to dissipate heat from electronic modules in medical imaging systems. Existing cooling apparatuses are formed from a cooling substrate configured to conduct heat and a pipe that is disposed on the cooling substrate and that is configured to transport a cooling liquid. By causing a heat-generating electronic module to abut against the cooling substrate, heat is transferred to the cooling substrate, and is then taken away by the cooling liquid in the pipe, to maintain a normal operating temperature of the electronic module.

However, because some electronic modules of the medical imaging system have special requirements (e.g., insulation, high thermal conductivity, and high temperature uniformity of a heat transfer surface, etc.) for the cooling apparatus, raw material for manufacturing the cooling substrate is very expensive. In addition, in the existing cooling apparatus, because the cooling substrate forms all structures except for the pipe for transporting the cooling liquid, it is necessary to use a large amount of raw materials to manufacture the cooling substrate, resulting in waste of the raw materials. In addition, in the existing cooling apparatus, the cooling substrate is a monolithic structure integrally formed from the raw materials, resulting in high manufacturing and transportation costs of the cooling substrate.

In view of at least one of the foregoing technical problems, embodiments of the present application provide a cooling apparatus for a medical imaging system, and a medical imaging system.

According to an aspect of the embodiments of the present application, a cooling apparatus for a medical imaging system is provided. The cooling apparatus comprises: a base; a cooling substrate, configured to conduct heat, the cooling substrate comprising at least one sub-substrate, and the at least one sub-substrate being disposed on the base; and a pipe, configured to transport a cooling liquid, at least a part of the pipe being disposed on the cooling substrate.

According to another aspect of the embodiments of the present application, the base is provided with at least one first through hole, the at least one sub-substrate is embedded in the at least one first through hole, and a first surface and a second surface opposite to the first surface of the sub-substrate are exposed from the base.

According to another aspect of the embodiments of the present application, the sub-substrate is in a one-to-one correspondence with the first through hole.

According to another aspect of the embodiments of the present application, a first surface of the sub-substrate is provided with a first groove for accommodating the pipe, and when the cooling substrate comprises a plurality of sub-substrates, ends of the first grooves of adjacent sub-substrates are aligned.

According to another aspect of the embodiments of the present application, a first surface of the base facing a same direction as the first surface of the sub-substrate is provided with a second groove for accommodating the pipe, and the second groove is aligned with an end of an adjacent first groove.

According to another aspect of the embodiments of the present application, when the cooling substrate comprises a plurality of sub-substrates, the plurality of sub-substrates have a same structure and size.

According to another aspect of the embodiments of the present application, when the cooling substrate comprises a plurality of sub-substrates, a gap between the plurality of sub-substrates is filled with a first thermally conductive material; or a gap between the cooling substrate and the base is filled with a first thermally conductive material; or a gap between the cooling substrate and the pipe is filled with a second thermally conductive material.

According to another aspect of the embodiments of the present application, the cooling apparatus is configured to dissipate heat from a detector module in the medical imaging system, wherein the detector module comprises at least one sub-module, and the sub-substrate of the cooling substrate is in a one-to-one correspondence with the sub-module of the detector module; or a plurality of sub-substrates of the cooling substrate correspond to one sub-module of the detector module; or one sub-substrate of the cooling substrate corresponds to a plurality of sub-modules of the detector module; or a plurality of sub-substrates of the cooling substrate correspond to a plurality of sub-modules of the detector module.

According to another aspect of the embodiments of the present application, the cooling apparatus is further configured to dissipate heat from a circuit board module in the medical imaging system, wherein the circuit board module and the detector module are respectively disposed on two sides of the cooling apparatus and abut against the cooling substrate of the cooling apparatus, a second through hole is formed in the sub-substrate of the cooling substrate, and the second through hole is configured to accommodate a connecting portion that connects the circuit board module and the detector module.

According to another aspect of the embodiments of the present application, the cooling substrate is an aluminum nitride substrate or an aluminum oxide substrate; or the base is a plastic base; or the pipe is a copper pipe, an aluminum pipe, or a stainless steel pipe.

According to another aspect of the embodiments of the present application, a medical imaging system is provided. The medical imaging system comprises the cooling apparatus according to any of the foregoing aspects.

One of the beneficial effects of the embodiments of the present application is that: The cooling apparatus comprises a base and a cooling substrate that is disposed on the base and that is configured to conduct heat. Compared with existing cooling apparatuses, the base is used to replace some of the structures of the cooling substrate. Therefore, the size of the cooling substrate can be reduced, thereby reducing the amount of a raw material used for manufacturing the cooling substrate, reducing a fault rate during manufacturing and transportation, and helping reduce the overall costs of the cooling apparatus.

With reference to the following description and drawings, specific implementations of the embodiments of the present application are disclosed in detail, and the way in which the principles of the embodiments of the present application can be employed are illustrated. It should be understood that the implementations of the present application are not limited in scope thereby. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a medical imaging system according to an embodiment of the present application;

FIG. 2 is a schematic diagram of a cooling apparatus according to an embodiment of the present application;

FIG. 3 is a schematic diagram of a base according to an embodiment of the present application;

FIG. 4 is another schematic diagram of a cooling apparatus according to an embodiment of the present application;

FIG. 5 is a schematic diagram of a sub-substrate according to an embodiment of the present application;

FIG. 6 is a schematic diagram of a cooling substrate according to an embodiment of the present application;

FIG. 7 is a schematic diagram of a division manner of a cooling substrate according to an embodiment of the present application;

FIG. 8 is a schematic diagram of an assembly process of a cooling apparatus according to an embodiment of the present application;

FIG. 9 is a schematic diagram of a detector assembly of a medical imaging system according to an embodiment of the present application; and

FIG. 10 is an exploded schematic diagram of a detector assembly of a medical imaging system according to an embodiment of the present application.

### DETAILED DESCRIPTION

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first” and “second” etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

In the descriptions of the present application, it should be noted that, unless otherwise specified and defined, the terms “connected” and “connect” should be understood in a broad sense, which, for example, may be a fixed connection, a detachable connection, or an integral connection; may be a mechanical connection or an electrical connection; may be a direct connection or an indirect connection by using an intermediate medium, or may be internal communication between two elements. Those of ordinary skill in the art may understand specific meanings of the foregoing terms in the present application according to a specific situation.

The features described and/or illustrated for one implementation may be used in one or more other implementations in the same or similar way, be combined with features in other implementations, or replace features in other implementations. The term “include/comprise” when used herein refers to the presence of features, integrated components, steps, or assemblies, but does not preclude the presence or addition of one or more other features, integrated components, steps, or assemblies.

A cooling apparatus according to the embodiments of the present application may be applied to various equipment, such as medical equipment, communication equipment, computer server equipment, etc. Using the application of the cooling apparatus to medical equipment as an example, the medical equipment includes, but is not limited to, magnetic resonance imaging (MRI) equipment, computed tomography (CT) equipment, ultrasound imaging equipment, positron emission computed tomography (PET) equipment, single photon emission computed tomography (SPECT) equipment, PET-CT, PET-MR, etc.

In the embodiments of the present application, the apparatus and system of the present application are exemplarily described by using a PET-MR scenario as an example. It should be understood that the content of the embodiments of the present application is also applicable to another medical imaging scenario.

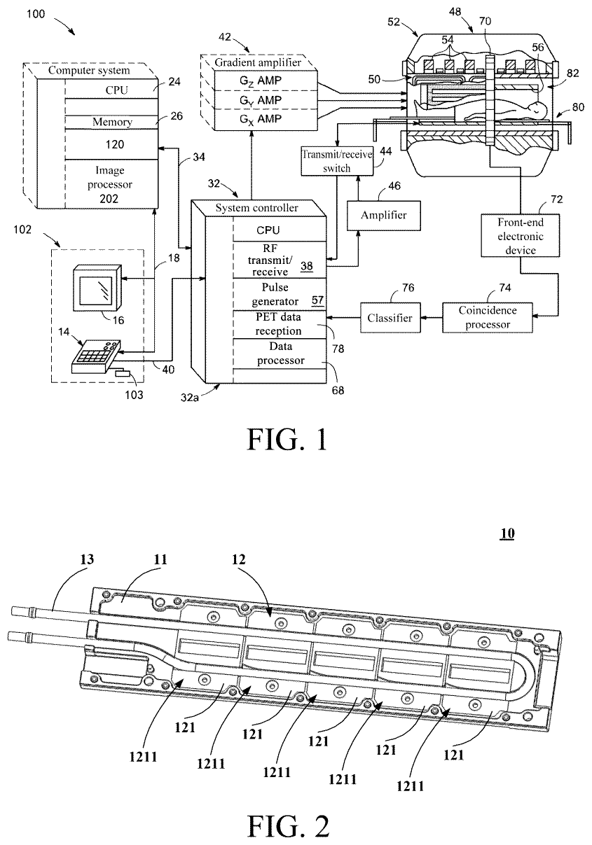

FIG. 1 is a schematic diagram of a medical imaging system according to an embodiment of the present application. A medical imaging system 100 is, for example, a PET-MR imaging system.

The system may be controlled from an operator console 102, and the operator console 102 includes a keyboard or another input apparatus 103, a control panel 14, and a display 16. The console 102 communicates with a separate computer system 120 by means of a link 18, and the computer system 120 enables an operator to control generation of an image and display of the image on the display 16. The computer system 120 includes multiple modules, such as an image processor module 202, a CPU module 24, and a memory module 26. The computer system 120 may be further connected to a permanent or back-up memory and a network, or may communicate with a separate system controller 32 by means of a link 34. The input apparatus 103 may include a mouse, a keyboard, a trackball, a touch-activated screen, a light pen, or any similar or equivalent input apparatus, and may be used for interactive geometry prescription.

The system controller 32 includes a set of modules that communicate with each other and that are connected to the operator console 102 by means of a link 40. The system controller 32 receives, by means of the link 34, a command indicating a scanning sequence or a plurality of scanning sequences to be performed. For MR data acquisition, an RF transmit/receive module 38 commands a scanner 48 to perform a desired scanning sequence by sending an instruction, a command, and/or a request that describes a plurality of RF pulse sequences to be generated and the timing, intensities, and shapes of the RF pulse sequences, so as to correspond to the timing and the length of a data acquisition window. A transmit/receive switch 44 controls the data to flow from the RF transmit module 38 to the scanner 48 through an amplifier 46 and flow from the scanner 48 to the RF receive module 38. The system controller 32 is further connected to a set of gradient amplifiers 42 to indicate the timing and the shape of a gradient pulse generated during a scan.

A gradient waveform instruction generated by the system controller 32 is sent to the gradient amplifiers 42 having Gx, Gy, and Gz amplifiers. The gradient amplifiers 42 may be located outside the scanner 48 or the system controller 32, or may be integrated therein. Each gradient amplifier excites a corresponding physical gradient coil in a gradient coil assembly substantially represented by 50, so as to generate a magnetic field gradient used to spatially encode an acquired signal. The gradient coil assembly 50 forms a part of a magnet assembly 52 that includes a polarizing magnet 54 and an RF coil assembly 56. Optionally, a gradient coil of the gradient coil assembly 50 may be independent of the magnet assembly 52. A coil of the RF coil assembly 56 may be used for both transmission and reception, or only for transmission or only for reception. A pulse generator 57 may be integrated into the system controller 32 as shown, or may be integrated into the scanner 48 to generate a pulse sequence signal for the gradient amplifiers 42 and/or the RF coil assembly 56 or more pulse sequence signals. In addition, the pulse generator 57 may generate PET data blanking signals synchronously with generation of the pulse sequences. These blanking signals may be generated on separate logic lines for subsequent data processing. An MR signal generated from an excitation pulse emitted by excited nuclei in a patient may be sensed by the RF coil assembly 56 and then transmitted to the RF transmit/receive module 38 through the transmit/receive switch 44. The MR signal is demodulated, filtered, and digitized in a data processor 68 of the system controller 32.

By means of an MR scan, one or more sets of raw k-space data are acquired in the data processor 68. The data processor 68 performs data transformation (by means of Fourier transform or another technique) on the raw k-space data to reconstruct image data. The image data is transmitted to the computer system 120 by means of the link 34, and is stored in the memory 26. In response to a command received from the operator console 102, the image data stored in the memory 26 may be archived in a long-term memory, or may be further processed by the image processor 202 or the CPU 24, transferred to the operator console 102, and presented on the display 16.

In a combined MR-PET scanning system, PET data may be acquired simultaneously with the MR data described above. Therefore, the scanner 48 further includes a detector array (or ring) 70 configured to detect gamma rays generated by annihilation of positrons emitted from a target. The detector array 70 preferably includes a plurality of scintillators and photovoltaic apparatuses arranged around a gantry. However, the detector array 70 may have any suitable structure for acquiring PET data. In addition, a scintillator package, the photovoltaic apparatuses, and other electronic devices of the detector array 70 do not need to be isolated from a magnetic field and/or an RF field applied by an MR component (the polarizing magnet 54 and the RF coil assembly 56). However, it is contemplated that embodiments of the present invention may include such isolation as is known in the art, or may be combined with various other isolation techniques.

The gamma rays detected by the detector array 70 are converted into electrical signals by the photovoltaic apparatuses of the detector array 70 and conditioned by a series of front-end electronic devices 72. These front-end electronic devices 72 may include various amplifiers, filters, and analog-to-digital converters. Digital signals outputted by the front-end electronic devices 72 are then processed by a coincidence processor 74 to match gamma ray detection as potential coincidence events. When two gamma rays strike detectors that are substantially opposite to each other, in the absence of random noise and an interaction between a signal and gamma ray detection, it is possible that positron annihilation has occurred somewhere along a line between the detectors. Therefore, coincidences determined by the coincidence processor 74 are classified as true coincidence events and finally integrated by a data classifier 76. Coincidence event data or PET data from the classifier 76 is received by the system controller 32 at a PET data reception port 78 and stored in the memory 26 for subsequent processing. A PET image may then be reconstructed by the image processor 202 and may be combined with an MR image to generate a hybrid structural and metabolic image or a functional image. The front-end electronic devices 72, the coincidence processor 74, and the classifier 76 may all be located outside the scanner 48 or the system controller 32, or may be integrated therein.

As further shown in FIG. 1, a patient support assembly 80 is included in the medical imaging system 100 to support the patient within the imaging system during data acquisition. The patient support assembly 80 extends into a main magnet bore 82 of the medical imaging system 100 and extends through the imaging system, so that the length of the patient support assembly 80 is substantially parallel to an axis of the bore 82. The patient support assembly 80 enables the patient to be moved relative to the medical imaging system 100 to various positions, including a loading position outside the bore of the medical imaging system 100 and at least one imaging position. When the patient is in the imaging position, at least a part of the patient is positioned in an imaging volume (that is, in the bore).

The embodiments of the present application are specifically described below.

Embodiments of the present application provide a cooling apparatus for a medical imaging system. FIG. 2 is a schematic diagram of a cooling apparatus according to an embodiment of the present application. As shown in FIG. 2, a cooling apparatus 10 includes a base 11, a cooling substrate 12 configured to conduct heat, and a pipe 13 configured to transport a cooling liquid. The cooling substrate 12 includes at least one sub-substrate 121, and the at least one sub-substrate 121 is disposed on the base 11. At least a part of the pipe 13 is disposed on the cooling substrate 12.

As described above, in existing cooling apparatuses, a structure corresponding to the base 11 and the cooling substrate 12 of the cooling apparatus 10 is a monolithic structure integrally formed from a raw material for manufacturing the cooling substrate 12. This results in waste of the raw material. In addition, due to the relatively large size of the monolithic structure, a fault rate during manufacturing and transportation is relatively high.

According to the foregoing embodiment of the present application, the cooling apparatus 10 includes a base 11 and a cooling substrate 12 that is disposed on the base 11 and that is configured to conduct heat. Compared with the existing cooling apparatuses, the base 11 is used to replace some structures of the cooling substrate. Therefore, the size of the cooling substrate 12 can be reduced, thereby reducing the amount of the raw material used for manufacturing the cooling substrate 12, reducing a fault rate during manufacturing and transportation, and helping reduce the overall costs of the cooling apparatus 10.

In some embodiments, in the cooling apparatus 10, the cooling substrate 12 may include a plurality of sub-substrates 121. By further dividing the cooling substrate 12 into the plurality of sub-substrates 121, modular design of the cooling substrate 12 can be performed, so that the size of each independent sub-substrate 121 is further reduced, thereby helping further reduce a fault rate during manufacturing and transportation and reduce manufacturing and transportation costs.

As shown in FIG. 2, the cooling substrate 12 includes five sub-substrates 121. The present application is not limited thereto, and the cooling substrate 12 may alternatively include another quantity of sub-substrates 121.

In addition, the cooling substrate 12 may alternatively include one sub-substrate 121. For example, one sub-substrate corresponding to the five sub-substrates 121 shown in FIG. 2 may be manufactured through integral forming.

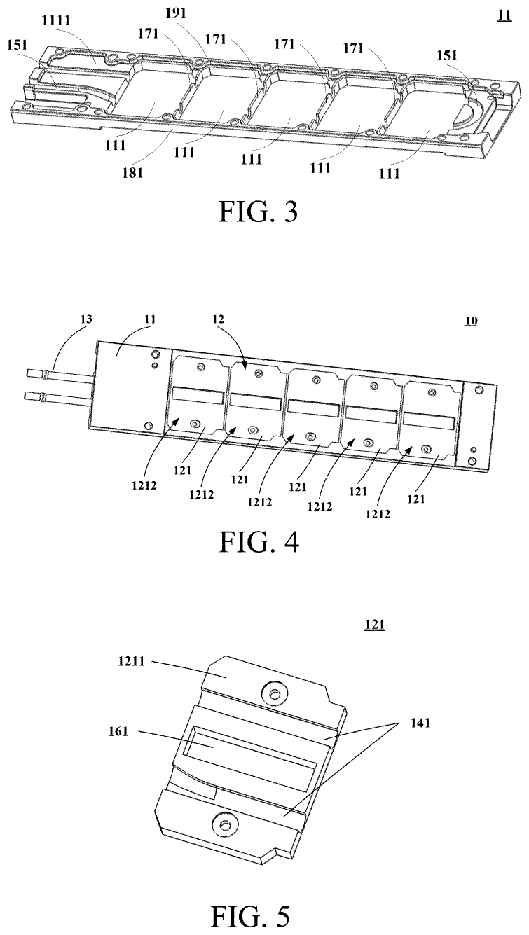

FIG. 3 is a schematic diagram of a base according to an embodiment of the present application. FIG. 4 is another schematic diagram of a cooling apparatus according to an embodiment of the present application, where a viewing direction in FIG. 4 is different from that in FIG. 2.

In some embodiments, as shown in FIG. 3, the base 11 is provided with at least one first through hole 111. As shown in FIG. 2 and FIG. 4, the at least one sub-substrate 121 of the cooling substrate 12 is embedded in the at least one first through hole 111, and a first surface 1211 and a second surface 1212 opposite to the first surface 1211 of the sub-substrate 121 are exposed from the base 11.

Therefore, by causing a heat-generating electronic module to abut against or be in contact with the first surface 1211 or the second surface 1212 of the sub-substrate 121, heat can be efficiently transferred to the sub-substrate 121.

In some embodiments, the sub-substrate 121 is in a one-to-one correspondence with the first through hole 111. That is, one sub-substrate 121 is disposed in one first through hole 111. Therefore, the sub-substrate 121 can be reliably affixed by the base 11. The present application is not limited thereto, and the sub-substrate 121 and the first through hole 111 may not be in a one-to-one correspondence, for example, a plurality of sub-substrates 121 may be disposed in one first through hole 111.

In some embodiments, a plurality of first through holes 111 (or sub-substrates 121) are uniformly spaced from each other. Therefore, a uniform heat dissipation effect can be provided.

In some embodiments, as shown in FIG. 3, a spacer plate 171 is disposed between adjacent first through holes. The spacer plate 171 may be in various forms.

For example, the height of the spacer plate 171 may be equal to the thickness of the sub-substrates 121. Therefore, strength of the base 11 can be ensured. The present application is not limited thereto, and the height of the spacer plate 171 may alternatively be less than the thickness of the sub-substrates 121.

For another example, as shown in FIG. 3, the base 11 includes a first frame 181 and a second frame 191 that are disposed opposite to each other, and the spacer plate 171 may be connected to the first frame 181 and the second frame 191. The present application is not limited thereto, and the spacer plate 171 may be only connected to the first frame 181 and disconnected from the second frame 191. Alternatively, the spacer plate 171 may be only connected to the second frame 191 and disconnected from the first frame 181. Alternatively, two ends of the spacer plate 171 are respectively connected to the first frame 181 and the second frame 191, and the spacer plate 171 is disconnected at a middle position. Therefore, adjacent first through holes can communicate with each other, so that adjacent sub-substrates 121 can be in contact with each other, or a gap between adjacent sub-substrates 121 is sufficiently small, thereby improving the heat dissipation effect.

In some embodiments, the base 11 may not be provided with the spacer plate 171. Therefore, adjacent sub-substrates 121 can be in contact with each other, or a gap between adjacent sub-substrates 121 is enabled to be sufficiently small, thereby improving the heat dissipation effect.

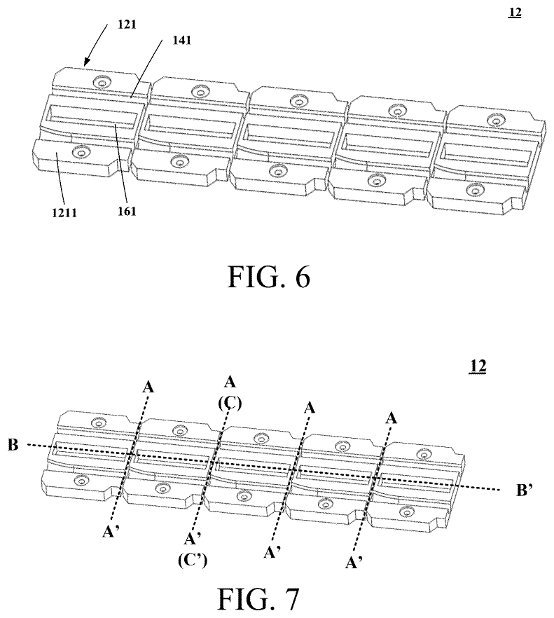

FIG. 5 is a schematic diagram of a sub-substrate according to an embodiment of the present application, and FIG. 6 is a schematic diagram of a cooling substrate according to an embodiment of the present application, where FIG. 5 shows one sub-substrate 121, and FIG. 6 shows a plurality of sub-substrates 121. In some embodiments, as shown in FIG. 5 and FIG. 6, the first surface 1211 of the sub-substrate 121 is provided with a first groove 141 for accommodating the pipe 13, and when the cooling substrate 12 includes a plurality of sub-substrates 121, ends of the first grooves 141 of adjacent sub-substrates 121 are aligned.

Therefore, a plurality of first grooves 141 located on the first surfaces 1211 of the plurality of sub-substrates 121 jointly form an integral groove for accommodating the pipe 13. Because the ends of adjacent first grooves 141 are aligned, the degree of bending of the integral groove for accommodating the pipe 13 can be prevented from being excessively large, thereby preventing the degree of bending of the pipe 13 from being excessively large, preventing the cooling liquid from accumulating in the pipe 13, and helping ensure a cooling effect.

In some embodiments, an accommodating channel formed by aligning the ends of the first grooves 141 of the plurality of sub-substrates 121 matches the shape of at least a part of the pipe 13. For example, as shown in FIG. 6, after the ends of the first grooves 141 of the plurality of sub-substrates 121 are aligned, a substantially linear accommodating channel is formed to accommodate a straight portion of the pipe 13. The present application is not limited thereto, and an accommodating channel in another shape may alternatively be formed after the ends of the first grooves 141 of the plurality of sub-substrates 121 are aligned.

In some embodiments, as shown in FIG. 3, a first surface 1111 of the base 11 facing a same direction as the first surface 1211 of the sub-substrate 121 is provided with a second groove 151 for accommodating the pipe 13. The second groove 151 is aligned with an end of an adjacent first groove 141.

Therefore, the base 11 and the cooling substrate 12 can be used to jointly accommodate the pipe 13, so that the pipe 13 can be more reliably affixed and the shape of the pipe 13 can be flexibly set. For example, an irregular shape of the pipe 13 can be disposed at a position corresponding to the base 11, and a regular shape of the pipe 13 can be disposed at a position corresponding to the cooling substrate 12. Therefore, the difficulty of manufacturing the cooling substrate 12 can be reduced, and manufacturing costs of the cooling substrate 12 are further reduced.

In addition, by aligning the second groove 151 of the base 11 with the end of the adjacent first groove 141, the degree of bending of a groove for accommodating the pipe 13 can be prevented from being excessively large, thereby preventing the degree of bending of the pipe 13 from being excessively large, preventing the cooling liquid from accumulating in the pipe 13, and helping ensure a cooling effect.

In some embodiments, when the cooling substrate 12 includes a plurality of sub-substrates 121, the plurality of sub-substrates 121 may have a same structure and size.

Therefore, it is only necessary to manufacture the sub-substrates 121 of one structure and size, which can reduce the difficulty of manufacturing the cooling substrate 12. In addition, when the sub-substrates 121 are assembled, it is not necessary to distinguish between the sub-substrates 121, which helps reduce manufacturing costs of the cooling substrate 12.

In addition, because the plurality of sub-substrates 121 have the same structure and size, it is convenient to reuse the sub-substrates 121, thereby further reducing costs. For example, when the shape of the electronic module that needs heat dissipation and that abuts against the cooling substrate 12 changes, the plurality of sub-substrates 121 of the cooling substrate 12 may be rearranged, so that the shape of the arranged sub-substrates 121 matches a new shape of the electronic module, and sub-substrates 121 do not need to be manufactured again.

In some embodiments, when the cooling substrate 12 includes a plurality of sub-substrates 121, a gap between the plurality of sub-substrates may be filled with a first thermally conductive material. In this way, the plurality of sub-substrates not only can exchange heat through the cooling liquid in the pipe 13, but also can exchange heat through the first thermally conductive material, thereby improving the temperature uniformity of the cooling substrate 12, improving the overall heat conduction efficiency of the cooling substrate 12, and improving the heat conduction effect.

The first thermally conductive material may be various materials having a heat conduction function. For example, the first thermally conductive material may be a thermally conductive epoxy resin, so that heat can be conducted between the sub-substrates 121 through the thermally conductive epoxy resin, and the plurality of sub-substrates 121 can be adhered and fixed. The present application is not limited thereto, and the first thermally conductive material may alternatively be another material.

In some embodiments, a gap between the cooling substrate 12 and the base 11 may be filled with the first thermally conductive material. For example, in a process of assembling the cooling apparatus 10, after the pipe 13 is disposed on the cooling substrate 12, the gap between the sub-substrates 121 and the gap between the cooling substrate 12 and the base 11 are uniformly filled with the first thermally conductive material, so that the cooling substrate 12 and the base 11 can be adhered and fixed, thereby improving the heat conduction effect.

In some embodiments, a gap between the cooling substrate 12 and the pipe 13 may be filled with a second thermally conductive material. Therefore, heat conduction efficiency between the cooling substrate 12 and the pipe 13 can be ensured, which helps improve the temperature uniformity of the cooling substrate 12, thereby improving the heat conduction effect.

The second thermally conductive material may be various materials having a heat conduction function. For example, the second thermally conductive material may be a tin material. The gap between the pipe 13 and the cooling substrate 12 is filled with the tin material by means of soldering, which not only can ensure a heat conduction effect, but also can reliably affix the pipe 13 and the cooling substrate 12. The present application is not limited thereto, and the second thermally conductive material may alternatively be another material.

In some embodiments, the cooling apparatus 10 may be used to dissipate heat from various electronic modules in the medical imaging system.

For example, the medical imaging system may include a detector module. An example in which the medical imaging system is a PET-MR imaging system is used, and the detector module is one of the core components of the PET-MR imaging system. The detector module may include a photoelectric conversion module. For example, the detector module converts rays emitted by a radionuclide into an electrical signal by means of the photoelectric conversion module, and then forms a distribution image of a metabolite substance. The photoelectric conversion module may include a photomultiplier tube (PMT), silicon photomultipliers (SiPM), and the like, which is not specifically limited in the present application.

An operating temperature of the photoelectric conversion module is one of the factors that affect the image quality of the PET-MR imaging system. In the embodiments of the present application, heat exchange by means of liquid cooling is used in the cooling apparatus 10 to reduce the operating temperature of the detector module, which helps improve the sensitivity of the detector module and improve the quality of an acquired image.

For example, the detector module abuts against a surface of the cooling substrate 12 of the cooling apparatus 10, heat is conducted from the detector module to the cooling substrate 12, and then the heat is taken away by the cooling liquid in the pipe 13, thereby reducing the operating temperature of the detector module.

In some embodiments, an arrangement manner of the sub-substrate 121 of the cooling substrate 12 may be related to the detector module. For example, the detector module may include at least one sub-module, and the sub-substrate 121 of the cooling substrate 12 may be in a one-to-one correspondence with the sub-module of the detector module. For example, one sub-substrate 121 abuts against one sub-module. The area of a surface of the sub-substrate 121 facing the sub-module may be equal to or greater than the area of a surface of the sub-module facing the sub-substrate 121, thereby reliably dissipating heat from the sub-module.

By causing the sub-substrate 121 of the cooling substrate 12 to be in a one-to-one correspondence with the sub-module of the detector module, the sub-substrate 121 of the cooling substrate 12 can be correspondingly arranged based on an arrangement manner of the sub-module of the detector module. That is, regardless of the arrangement manner of the sub-module of the detector module, heat can be dissipated from the detector module through the cooling substrate 12 including the sub-substrate 121. In addition, because the sub-substrate 121 of the cooling substrate 12 is in a one-to-one correspondence with the sub-module of the detector module, it is convenient to determine whether a certain sub-substrate 121 is faulty through temperature measurement, and when a fault occurs, only the sub-substrate 121 needs to be replaced, and the entire cooling substrate 12 does not need to be replaced, thereby reducing maintenance costs.

The present application is not limited thereto, and another correspondence may alternatively be used between the sub-substrate 121 of the cooling substrate 12 and the sub-module of the detector module. In other words, the cooling substrate 12 may be divided into the plurality of sub-substrates 121 in another manner.

For example, the plurality of sub-substrates 121 of the cooling substrate 12 may correspond to one sub-module of the detector module.

For example, one sub-substrate 121 of the cooling substrate 12 may correspond to a plurality of sub-modules of the detector module.

For example, a plurality of sub-substrates 121 of the cooling substrate 12 may correspond to a plurality of sub-modules of the detector module.

FIG. 7 is a schematic diagram of a division manner of a cooling substrate according to an embodiment of the present application.

As shown in FIG. 7, the cooling substrate 12 may be divided into five sub-substrates along four dashed lines AA′, where the five sub-substrates respectively correspond to five sub-modules of the detector module.

Alternatively, as shown in FIG. 7, the cooling substrate 12 may be divided into two sub-substrates along a dashed line CC′, where the left sub-substrate corresponds to two sub-modules of the detector module, and the right sub-substrate corresponds to three sub-modules of the detector module.

Alternatively, as shown in FIG. 7, the cooling substrate 12 may be divided into two sub-substrates along a dashed line BB′, where the upper and lower sub-substrates correspond to five sub-modules of the detector module.

Alternatively, as shown in FIG. 7, the cooling substrate 12 may be divided into a plurality of sub-substrates along various combinations of the plurality of foregoing dashed lines. For example, the substrate is divided into 10 sub-substrates along the four dashed lines AA′ and the dashed line BB′, where every two sub-substrates correspond to one sub-module of the detector module. The present application is not limited thereto, and the cooling substrate 12 may alternatively be divided into sub-substrates in another division manner.

In some embodiments, the medical imaging system may further include a circuit board module, and the circuit board module may be connected to the detector module of the medical imaging system. The circuit board module may include a digital-to-analog conversion module, and the digital-to-analog conversion module is configured to perform digital-to-analog conversion on a first signal inputted to the detector module or a second signal outputted from the detector module.

The cooling apparatus 10 in the embodiments of the present application may further dissipate heat from the circuit board module. For example, the circuit board module and the detector module may be respectively disposed on two sides of the cooling apparatus 10 and abut against the cooling substrate 12 of the cooling apparatus 10. As shown in FIG. 5 and FIG. 6, a second through hole 161 is formed in the sub-substrate 121 of the cooling substrate 12, and the second through hole 161 is configured to accommodate a connecting portion that connects the circuit board module and the detector module.

In some embodiments, the cooling substrate 12 may be a substrate made of various materials. For example, the cooling substrate 12 may be an aluminum nitride substrate (an ALN substrate). Due to characteristics of an aluminum nitride material, the aluminum nitride substrate can meet requirements of insulation, high thermal conductivity, high temperature uniformity of a heat transfer surface, and the like, thereby ensuring the cooling performance of the cooling apparatus 10. The present application is not limited thereto, and the cooling substrate 12 may alternatively be an aluminum oxide substrate (an ALO substrate). Compared with the aluminum nitride substrate, a heat dissipation effect of the aluminum oxide substrate is inferior to that of the aluminum nitride substrate, but costs of the aluminum oxide substrate are much lower than those of the aluminum nitride substrate.

In some embodiments, the base 11 may be a base made of various materials. For example, the base 11 is a plastic base or the like. For example, the base 11 may be a polyoxymethylene (POM) base or the like. Therefore, the strength and toughness of the base 11 can be ensured, thereby helping improve the overall reliability of the cooling apparatus 10.

In some embodiments, the pipe 13 may be a pipe made of various materials. For example, the pipe 13 is a copper pipe, an aluminum pipe, a stainless steel pipe, or the like.

The following exemplarily describes an assembly manner of the cooling apparatus 10 in the embodiments of the present application with reference to the accompanying drawings.

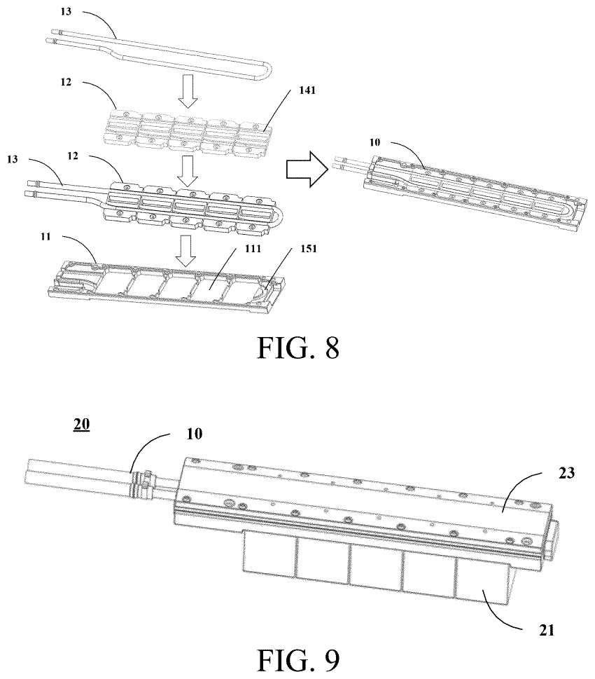

FIG. 8 is a schematic diagram of an assembly process of a cooling apparatus according to an embodiment of the present application. As shown in FIG. 8, the pipe 13 is fixedly connected in the first groove 141 of the cooling substrate 12 by soldering, and the gap between the pipe 13 and the cooling substrate 12 is filled with the tin material. The pipe 13 and the cooling substrate 12 are embedded in the base 11, for example, a part of the pipe 13 is disposed in the second groove 151 of the base 11, and the cooling substrate 12 is disposed in the first through hole 111 of the base 11. The gap between the sub-substrates 121 of the cooling substrate 12 and the gap between the cooling substrate 12 and the base 11 are filled with the thermally conductive epoxy resin, thereby assembling the cooling apparatus 10. The present application is not limited thereto, and the foregoing assembly steps may be in another order, or the cooling apparatus 10 may be assembled in another manner.

The following exemplarily describes an application manner of the cooling apparatus 10 in the embodiments of the present application with reference to the accompanying drawings.

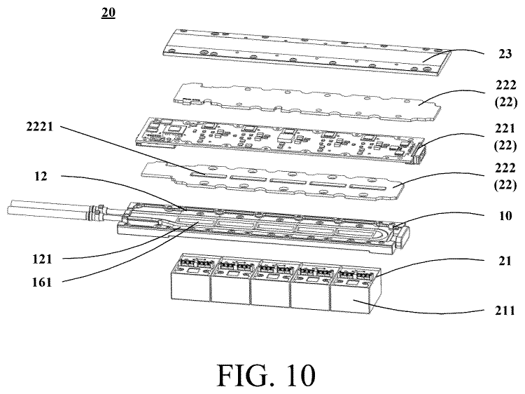

FIG. 9 is a schematic diagram of a detector assembly of a medical imaging system according to an embodiment of the present application, and FIG. 10 is an exploded schematic diagram of a detector assembly of a medical imaging system according to an embodiment of the present application. As shown in FIG. 9 and FIG. 10, the cooling apparatus 10 in the embodiments of the present application is applied to a detector assembly 20.

As shown in FIG. 9 and FIG. 10, the detector assembly 20 includes the cooling apparatus 10 and a detector module 21. As shown in FIG. 10, the detector module 21 includes five sub-modules 211 arranged in a row. The cooling substrate 12 of the cooling apparatus 10 includes five sub-substrates 121 respectively corresponding to the sub-modules 211, and in an assembled state, the sub-modules 211 abut against the second surfaces (lower surfaces in FIG. 10) of the sub-substrates 121, thereby cooling the sub-modules 211.

As shown in FIG. 10, the detector module 21 further includes a circuit board module 22. The circuit board module 22 includes a circuit board 221 and heat conduction plates 222 disposed on two sides of the circuit board 221. In the assembled state, a heat conduction plate 222 of the circuit board module 22 abuts against the first surfaces (upper surfaces in FIG. 10) of the five sub-substrates 121, thereby cooling the circuit board module 22.

As shown in FIG. 10, in the cooling apparatus 10, the second through hole 161 is formed in each sub-substrate 121 of the cooling substrate 12 and is configured to accommodate the connecting portion that connects the detector module 21 and the circuit board module 22. The connecting portion may be a part of the detector module 21. The present application is not limited thereto, and the connecting portion may alternatively be a part of the circuit board module 22, or the connecting portion may be a component independent of the detector module 21 and the circuit board module 22.

As shown in FIG. 10, a third through hole 2221 is formed in the heat conduction plate 222 of the circuit board module 22 that abuts against the cooling substrate 12 of the cooling apparatus 10. The third through hole 2221 is disposed opposite to the second through hole 161, and is configured to accommodate the connecting portion that connects the detector module 21 and the circuit board module 22.

In some embodiments, as shown in FIG. 9 and FIG. 10, the detector assembly 20 further includes a housing 23, and the housing 23 is configured to cover internal components of the circuit board module 22.

It should be noted that only the components or modules related to the present embodiment are described above. The present application is not limited thereto, and the cooling apparatus may further include other components or modules. For specific content related to these components or modules, reference may be made to the related art.

In addition, for the sake of simplicity, the connection relationships and positional relationships between various components or modules are only exemplarily shown in the above drawings, the present application is not limited thereto, and other connection relationships and positional relationships may alternatively be used between the foregoing components or modules. The specific shapes of the pipe and the grooves for accommodating the pipe are only exemplarily shown in the above drawings, the present application is not limited thereto, and the pipe and the grooves for accommodating the pipe may alternatively be in other shapes.

According to the foregoing embodiments, the cooling apparatus includes a base and a cooling substrate that is disposed on the base and that is configured to conduct heat. Compared with existing cooling apparatuses, the base is used to replace some of the structures of the cooling substrate. Therefore, the size of the cooling substrate can be reduced, thereby reducing the amount of a raw material used for manufacturing the cooling substrate, reducing a fault rate during manufacturing and transportation, and helping reduce the overall costs of the cooling apparatus.

Embodiments of the present application further provide a medical imaging system. The configuration of the medical imaging system is as shown in FIG. 1, and repeated portions will not be described again. The medical imaging system according to the embodiments of the present application differs from the foregoing medical imaging system in FIG. 1 in that the medical imaging system according to the embodiments of the present application includes the foregoing cooling apparatus.

In some embodiments, the medical imaging system further includes a detector module. The detector module may include a photoelectric conversion module. For example, the detector module is included in the detector array 70 in the medical imaging system 100 shown in FIG. 1.

The cooling apparatus is configured to dissipate heat from the detector module. The detector module includes at least one sub-module. The sub-substrate of the cooling substrate is in a one-to-one correspondence with the sub-module of the detector module; or a plurality of sub-substrates of the cooling substrate correspond to one sub-module of the detector module; or one sub-substrate of the cooling substrate corresponds to a plurality of sub-modules of the detector module; or a plurality of sub-substrates of the cooling substrate correspond to a plurality of sub-modules of the detector module.

In some embodiments, the medical imaging system further includes a circuit board module. The circuit board module may include a digital-to-analog conversion module, and the digital-to-analog conversion module is electrically connected to the photoelectric conversion module of the detector module.

The cooling apparatus is further configured to dissipate heat from the circuit board module. The circuit board module and the detector module are respectively disposed on two sides of the cooling apparatus and abut against the cooling substrate of the cooling apparatus.

According to the foregoing embodiment, the medical imaging system includes a cooling apparatus, the cooling apparatus includes a base and a cooling substrate that is disposed on the base and that is configured to conduct heat. Compared with existing cooling apparatuses, the base is used to replace some of the structures of the cooling substrate. Therefore, the size of the cooling substrate can be reduced, thereby reducing the amount of a raw material used for manufacturing the cooling substrate, reducing a fault rate during manufacturing and transportation, helping reduce the overall costs of the cooling apparatus, and further helping reduce the overall costs of the medical imaging system.

The foregoing apparatus, system, and method of the present application may be implemented by hardware, or may be implemented by hardware in combination with software. The present application relates to such a computer-readable program that when executed by a logic component, the program causes the logic component to implement the foregoing apparatus and system, or a constituent component, or causes the logic component to implement various methods or steps as described above. The present application further relates to a storage medium for storing the above program, such as a hard disk, a disk, an optical disk, a DVD, a flash memory, etc.

The method/apparatus/system described with reference to the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory apparatus, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for executing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific implementations. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A cooling apparatus for a medical imaging system, characterized by comprising:
a base;
a cooling substrate, configured to conduct heat, the cooling substrate comprising at least one sub-substrate, and the at least one sub-substrate being disposed on the base; and
a pipe, configured to transport a cooling liquid, at least a part of the pipe being disposed on the cooling substrate.

2. The cooling apparatus according to claim 1, wherein
the base is provided with at least one first through hole, the at least one sub-substrate is embedded in the at least one first through hole, and a first surface and a second surface opposite to the first surface of the sub-substrate are exposed from the base.

3. The cooling apparatus according to claim 2, wherein the sub-substrate is in a one-to-one correspondence with the first through hole.

4. The cooling apparatus according to claim 1, wherein
a first surface of the sub-substrate is provided with a first groove for accommodating the pipe, and when the cooling substrate comprises a plurality of sub-substrates, ends of the first grooves of adjacent sub-substrates are aligned.

5. The cooling apparatus according to claim 4, wherein
a first surface of the base facing a same direction as the first surface of the sub-substrate is provided with a second groove for accommodating the pipe, and the second groove is aligned with an end of an adjacent first groove.

6. The cooling apparatus according to claim 1, wherein
when the cooling substrate comprises a plurality of sub-substrates, the plurality of sub-substrates have a same structure and size.

7. The cooling apparatus according to claim 1, wherein
when the cooling substrate comprises a plurality of sub-substrates, a gap between the plurality of sub-substrates is filled with a first thermally conductive material; or,
a gap between the cooling substrate and the base is filled with a first thermally conductive material; or,
a gap between the cooling substrate and the pipe is filled with a second thermally conductive material.

8. The cooling apparatus according to claim 1, wherein
the cooling apparatus is configured to dissipate heat from a detector module in the medical imaging system, wherein the detector module comprises at least one sub-module, and
the sub-substrate of the cooling substrate is in a one-to-one correspondence with the sub-module of the detector module; or,
a plurality of sub-substrates of the cooling substrate correspond to one sub-module of the detector module; or,
one sub-substrate of the cooling substrate corresponds to a plurality of sub-modules of the detector module; or,
a plurality of sub-substrates of the cooling substrate correspond to a plurality of sub-modules of the detector module.

9. The cooling apparatus according to claim 8, wherein
the cooling apparatus is further configured to dissipate heat from a circuit board module in the medical imaging system, wherein the circuit board module and the detector module are respectively disposed on two sides of the cooling apparatus and abut against the cooling substrate of the cooling apparatus, a second through hole is formed in the sub-substrate of the cooling substrate, and the second through hole is configured to accommodate a connecting portion that connects the circuit board module and the detector module.

10. The cooling apparatus according to claim 1, wherein
the cooling substrate is an aluminum nitride substrate or an aluminum oxide substrate; or,
the base is a plastic base.

11. A medical imaging system, characterized by comprising the cooling apparatus according to claim 1.
